(19) United States
(12) Patent Application Publication
(10) Pub. No.: US 2024/0238318 A1
(43) Pub. Date: Jul. 18, 2024

(54) L-FUCOSE AND ANTI-ANDROGEN RECEPTOR THERAPY FOR TREATMENT OF CANCER

(71) Applicant: H. LEE MOFFITT CANCER CENTER AND RESEARCH INSTITUTE, INC., Tampa, FL (US)

(72) Inventor: Eric K. LE-LAU, Tampa, FL (US)

(21) Appl. No.: 18/559,204

(22) PCT Filed: May 6, 2022
(86) PCT No.: PCT/US2022/028022

§ 371 Date: Nov. 6, 2023

Related U.S. Application Data
(60) Provisional application No. 63/184,937, filed on May 6, 2021.

(51) Int. Cl. A61K 31/7004; A61K 31/167; A61K 31/277; A61K 31/4166; A61K 31/4439; A61K 31/496; A61K 31/573; A61K 31/58; A61K 38/09; A61K 39/00; A61K 39/395; G01N 33/574

(52) U.S. Cl. CPC A61K 31/7004; A61K 31/167; A61K 31/277; A61K 31/4166; A61K 31/4439; A61K 31/496; A61K 31/573; A61K 31/58; A61K 38/09; A61K 39/3955; A61K 39/4611; A61K 39/4613; A61K 39/4631; G01N 33/57484; G01N 2333/723; G01N 2333/91102; G01N 2440/38

(57) ABSTRACT

Disclosed are methods for treating, inhibiting, reducing, and/or preventing cancers (such as, for example melanoma) and/or metastasis (such as, for example metastatic melanoma) comprising administering to a subject a L-fucose and an anti-androgen therapy.

This application claims the benefit of U.S. Provisional Application No. 63/184,937, filed on May 6, 2021, which is incorporated herein by reference in its entirety.

This invention was made with government support under Grant No. CA241559 awarded by National Institutes of Health. The government has certain rights in the invention.

### I. BACKGROUND

Melanoma is one of the most lethal skin cancers worldwide, characterized by a striking ability to metastasize and develop therapeutic resistance. There are also sex-dependent disparities in the incidence and mortality of melanoma with the lethality rate of melanoma being nearly twice as high for male patients when compared to female patients.

Typically, the immune system plays a crucial role in recognizing and suppressing cancers in the body. Unfortunately, melanomas can interact with and inactivate immune cells. Currently, among the most effective anti-melanoma therapies is immunotherapies. These include antibody-based immunotherapies, such Nivolumab or Ipilumumab, which block these inhibitory interactions, “reactivating” the tumor-suppressing activities of immune cells, as well as adoptive cell (“TIL”) therapy which involves the ex vivo expansion of tumor-infiltrating lymphocytes. However, despite recent successes of such immunotherapies, responsiveness (and durations of responses) is limited to subsets of patients.

Despite reports of striking efficacy, durable responses of immunotherapies have been limited to subsets of patients. In attempt to improve responses, clinical trials have tested combinations of immunotherapies with other therapeutic interventions, with limited success 1. Unfortunately, patients often experience significant adverse events, sometimes resulting in their withdrawal from the clinical trial. Another ongoing challenge with immunotherapies is ineffective patient stratification. What are needed are new immunotherapies that can overcome the limitations of existing therapeutic protocols.

### II. SUMMARY

Disclosed are methods related to enhancing immune responses and treating cancers with the administration of fucose.

In one aspect, disclosed herein are methods of detecting the presence of a cancer and/or metastasis (such as, for example, melanoma, prostate cancer, or breast cancer) in a subject comprising obtaining a cancerous tissue sample from the subject and assaying the level of androgen receptor (AR), Fucosyltransferase 4 (FUT4), fucosylated L1CAM, and/or FUT4 fucosylated/-regulated glycans and/or proteoglycans in the sample, wherein the presence of or an increase in the level of androgen receptor (AR), Fucosyltransferase 4 (FUT4), fucosylated L1CAM, and/or FUT4 fucosylated/-regulated glycans and/or proteoglycans relative to a control indicates the presence of a cancer and/or metastatic cancer. In some aspect, where an increase in AR), Fucosyltransferase 4 (FUT4), fucosylated L1CAM, and/or FUT4 fucosylated/-regulated glycans and/or proteoglycans relative to a control, the method can further comprise treating the subject with anti-androgen therapy in combination with an agent that increases the amount of fucosylation (such as, for example, L-fucose, D-fucose, fucose-1-phosphate, or GDP-L-fucose).

Also disclosed herein are methods of measuring the severity of a cancer and/or metastasis (such as, for example, melanoma, prostate cancer, or breast cancer) in a subject comprising obtaining a cancerous tissue sample from the subject and assaying the level of androgen receptor (AR), Fucosyltransferase 4 (FUT4), fucosylated L1CAM, and/or FUT4 fucosylated/-regulated glycans and/or proteoglycans in the sample relative to a control directly correlates with the severity of the cancer and/or metastasis. Where a severe cancer or metastasis is detected, the method can further comprise treating the subject with anti-androgen therapy in combination with an agent that increases the amount of fucosylation (such as, for example, L-fucose, D-fucose, fucose-1-phosphate, or GDP-L-fucose).

In one aspect, disclosed herein are methods of selecting cancer patients for anti-androgen therapy comprising obtaining a cancerous tissue sample from the patient and measuring the level of androgen receptor (AR), Fucosyltransferase 4 (FUT4), fucosylated L1CAM, and/or FUT4 fucosylated/-regulated glycans and/or proteoglycans in the tissue sample, wherein an increase in androgen receptor (AR), Fucosyltransferase 4 (FUT4), fucosylated L1CAM, and/or FUT4 fucosylated/-regulated glycans and/or proteoglycans relative to noncancerous tissue indicates that the patient should receive anti-androgen therapy in combination with an agent that increases the amount of fucosylation (such as, for example, L-fucose, D-fucose, fucose-1-phosphate, or GDP-L-fucose).

In one aspect, disclosed herein are methods of treating a treating, inhibiting, decreasing, reducing, ameliorating, and/or preventing a cancer and/or metastasis (such as, for example, melanoma, prostate cancer, or breast cancer) in a subject comprising administering to the subject an agent that increases the amount of fucosylation (such as, for example, L-fucose, D-fucose, fucose-1-phosphate, or GDP-L-fucose) and an anti-androgen therapy (anti-androgen therapies including but not limited to luteinizing hormone-releasing hormone (LHRH) agonists (such as, for example, leuprolide, goserelin, triptorelin, and/or histrelin), LHRH antagonist (such as, for example degarelix and/or relugolix), and/or anti-androgen therapy (such as, for example, bicalutamide, nilutamide, flutamide, abiraterone, corticosteroids, ketoconazole, and/or apalutamide). In some aspect, the fucose is administered before, after, concurrent with, and/or simultaneous to the administration of the anti-androgen therapy.

Also disclosed herein are methods of treating a treating, inhibiting, decreasing, reducing, ameliorating, and/or preventing a cancer and/or metastasis in a subject of any preceding aspect, further comprising administering to the subject an immune checkpoint blockade inhibitor (such as, for example, PD-1 inhibitors lambrolizumab, OPDIVO® (Nivolumab), KEYTRUDA® (pembrolizumab), and/or pidilizumab; the PD-L1 inhibitors BMS-936559, TECENTRIQ® (Atezolizumab), IMFINZI® (Durvalumab), and/or BAVENCIO® (Avelumab); and/or the CTLA-4 inhibitor YERVOY (ipilimumab)). In one aspect, the fucose increasing agent is administered before and/or contiguous with administration of the immune checkpoint inhibitor.

In one aspect, disclosed herein are methods of treating a treating, inhibiting, decreasing, reducing, ameliorating, and/or preventing a cancer and/or metastasis (such as, for example, melanoma, prostate cancer, or breast cancer) in a subject of any preceding aspect, further comprising administering to the subject an adoptive cell therapy (such as, for example the transfer of tumor infiltrating lymphocytes (TILs), tumor infiltrating NK cells (TINKs), dendritic cell (DC), marrow infiltrating lymphocytes (MILs), chimeric antigen receptor (CAR) T cells, and/or CAR NK cells).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D and 1E show that Melanoma cells express androgen-inducible and transcriptionally active AR. FIG. 1A shows the AR expression status in male and female melanoma patients from TCGA skin cutaneous melanoma (SKCM) dataset. FIG. 1B shows the expression of AR in primary and metastatic melanoma patients from TCGA SKCM dataset. FIG. 1C shows immunoblotting analysis for baseline AR protein level across 10 melanoma cell lines. LNCaP prostate cancer cell line serves as a positive control for AR expression. FIG. 1D shows nuclear fractionation followed by immunoblotting of AR level in WM793 cells treated with 100 nM dihydrotestosterone (DHT) over 96 hours. FIG. 1E shows an AR binding motif-containing promoter (ARR2) luciferase assay on WM793 cells+100 nM DHT.

FIGS. 2A, 2B, 2C, and 2D show the functional effects of androgen on melanoma cells. FIG. 2A shows an MTT assay with WM793 cells+100 nM DHT (left) or +10 uM AR inhibitor (AZD3514) (right). FIG. 2B shows BrdU staining and (2C) Wound healing assay with WM793 cells+100 nM DHT. FIG. 2D shows a growth curve of BRAF-mutant SM1 tumors in C57BL6 mice. Mice were castrated or not ˜ 1.5 weeks prior to injection.

FIGS. 3A, 3B, 3C, and 3D show that AR transcriptionally upregulates FUT4 expression via binding to the ARE motif in FUT4 promoter. FIG. 3A shows predicted AR-binding sites in the promoter of FUT4 (SEQ ID NO: 1), FUT1 (SEQ ID NO: 2), SLC35C2 (SEQ ID NO: 3), and FUK (SEQ ID NO: 4) genes. FIG. 3B shows qRT-PCR assessing mRNA levels of FUK and FUT4 altered by DHT treatment in WM793 cells. FIG. 3C shows ChIP-qPCR analysis of the enrichment of AR protein at −515-502 bp promoter region of FUT4 gene upon DHT treatment. FIG. 3D shows Hallmark GSEA associates FUT4 expression with androgen response gene signatures in TCGA SKCM samples.

FIG. 4 shows global fucosylation is lower in males than females.

FIGS. 5A and 5B show that AR-FUT4-dependent signaling regulates cell adhesion/motility, whereas AR-dependent/FUT4-independent signaling regulates cell division. FIG. 5A shows phospho-proteomics profiling of EV/FUT4-OE melanoma cells±AR inhibitor. FIG. 5B (left) shows ingenuity pathway analysis (IPA) listed adherens junctions (AJs) as the top 1 AR/FUT4-regulated signaling. FIG. 5B (right) shows AJs structure diagram. Red circles denote hits from our profiles.

FIGS. 6A, 6B, 6C, 6D, and 6E show that the AR-FUT4 axis facilitates melanoma invasion via disrupting N-Cadherin/catenin junction complexes. FIG. 6A shows a clonogenic assay of WM793 cells±10 uM AR inhibitor. FIG. 6B shows matrigel invasion assay on EV/FUT4-OE WM793 cells±10 uM AR inhibitor treatment. FIG. 6C shows 3D spheroid cell invasion assay on EV/FUT4-OE WM793 cells±AR inhibitor. FIG. 6D shows proximity ligation assay of the Interaction between N-cadherin and catenin proteins in EV/FUT4-OE WM793 cells and parental WM793 cells±10 uM AR inhibitor. FIG. 6E (left) shows the expression of FUT4 in primary and metastatic melanoma patients from TCGA SKCM dataset. FIG. 6E (right) shows Hallmark GSEA associates FUT4 expression with epithelial-mesenchymal transition (EMT) gene signatures in TCGA SKCM samples.

FIG. 7 shows the results of a knockdown experiment showing that knockdown of FUT4 abrogates androgen stimulated melanoma activity.

FIG. 8 shows a diagram of melanoma cells expressing androgen-responsive AR and the effect this has on melanoma proliferation and migration through FUT4.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A number of embodiments of the invention have been described. Nevertheless, it will be understood that various modifications may be made without departing from the spirit and scope of the invention. Accordingly, other embodiments are within the scope of the following claims.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims that follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “biological sample” or “tissue sample” refers to any portion of biological material from a subject to be used in any of the methods or as a part of any of the compositions disclosed herein including, but not limited to, tissue biopsy, whole blood, serum, plasma, peripheral blood mononuclear cells, urine sample, lung lavage, sputum, saliva, cerebrospinal fluid, and fecal sample. The biological can include samples for normal and cancerous tissue. Sample may be obtained from any tissue a subject by any means known in the art (tissue resection, biopsy phlebotomy, core biopsy).

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, fucoidan, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

A “binding molecule” or “antigen binding molecule” (e.g., an antibody or antigen-binding fragment thereof) as provided herein refers in its broadest sense to a molecule that specifically binds an antigenic determinant. In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods and Compositions

Although less than in prostate cancer cells, melanoma cells express variable levels of functional androgen receptor, seemingly regardless of mutational background. Among many prognostic factors that have been studied, sex has emerged as one of the most intriguing but least understood independent indicators for melanoma outcomes, which male patients present disadvantages in tumor prognosis and survival status. We discovered an unexpected correlation between sex and melanoma fucosylation, the post-translational modification of glycoproteins with the dietary sugar L-fucose (L-fuc). Fucosylation can promote or suppress tumors-divergent functions dictated by 13 tumor-promoting or tumor-suppressing fucosyltransferases (FUTs) that conjugate fucose moieties onto targeted proteins. Male melanoma patients, who have poorer prognosis, exhibit lower intratumor global fucosylation levels, suggesting that androgen signaling might globally reduce but shift the functional balance of fucosylation towards tumor promotion. However, there is currently a knowledge gap regarding the association between androgen/AR signaling and fucosylation in melanoma. We discovered that androgen/AR increases tumorigenic fucosylation by transcriptionally upregulating an oncogenic FUT, FUT4, which is known to synthesize structural subtypes of fucosylation onto cell surface proteins that can modulate cell-cell adhesion, metastasis and therapy resistance. Herein we show a new functional role for AR signaling in driving aggressive pathogenesis in male melanoma patients by promoting FUT4-mediated, pro-tumorigenic forms of fucosylation.

Fucosylation, the post-translational modification of proteins with the dietary sugar L-fucose, is a mechanism that is well established for its importance in immune cell biology and organ developmental processes but that is poorly understood in terms of its roles in cancer. Fucose is transported extracellularly through the plasma membrane. In the cytosol, free L-Fucose is phosphorylated by fucokinase (FUK) and GDP-coupled by fucose-1-phosphate guanylyltransferase (FPGT) to yield GDP-fucose, which is the global substrate for cellular protein fucosylation. Synthesized GDP-fucose is transported into ER/Golgi via SLC35C1/2 transporters, where fucose moieties are conjugated onto proteins by 13 FUTs. FUK, FPGT, and SLC35C1/2 regulate global substrate (GDP-fucose) availability, whereas the FUTs are rate-limiting and determine the tumor-promoting vs. tumor-suppressive subtype of fucosylation.

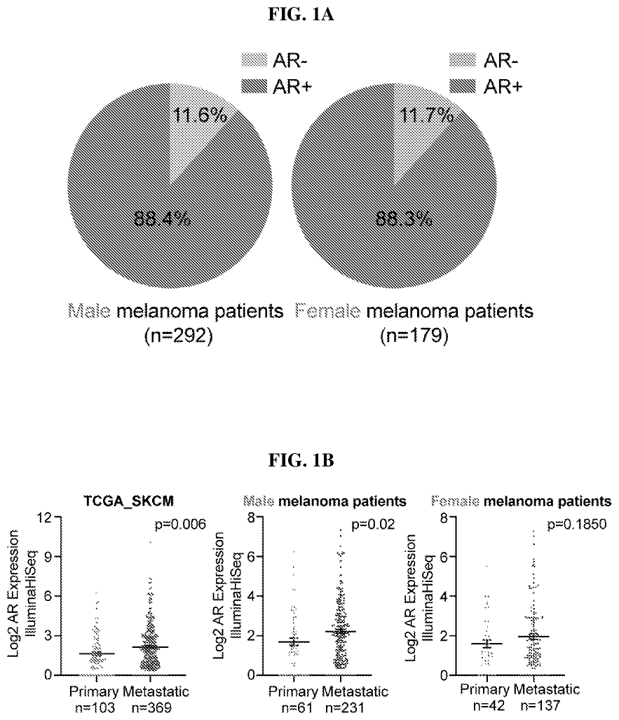

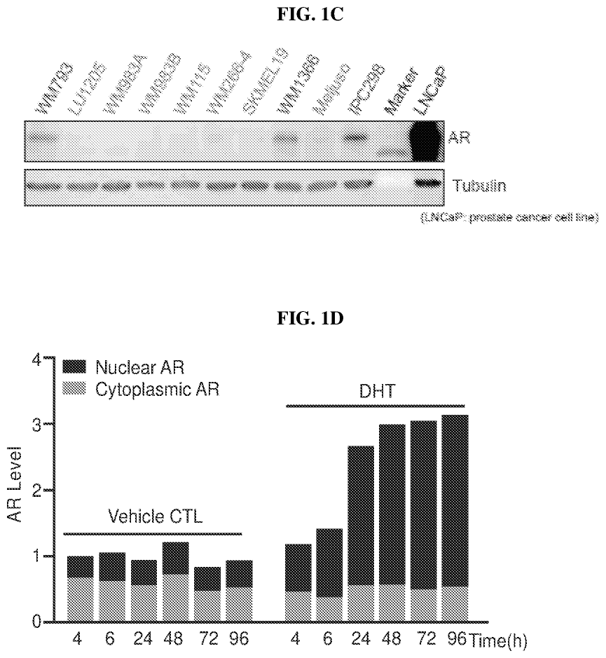

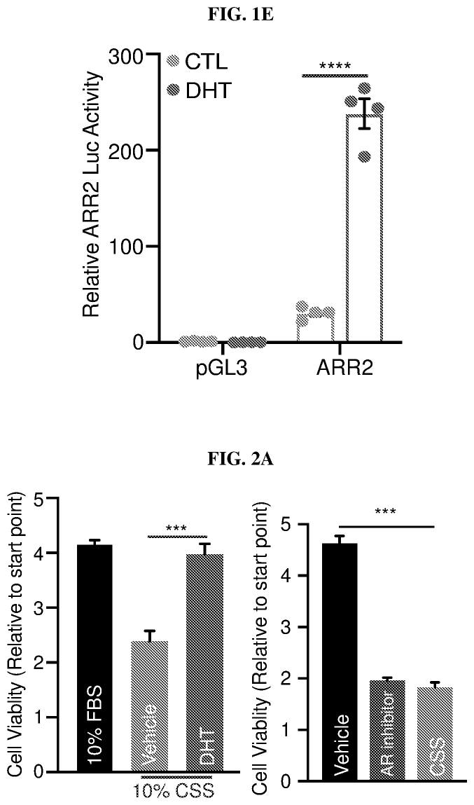

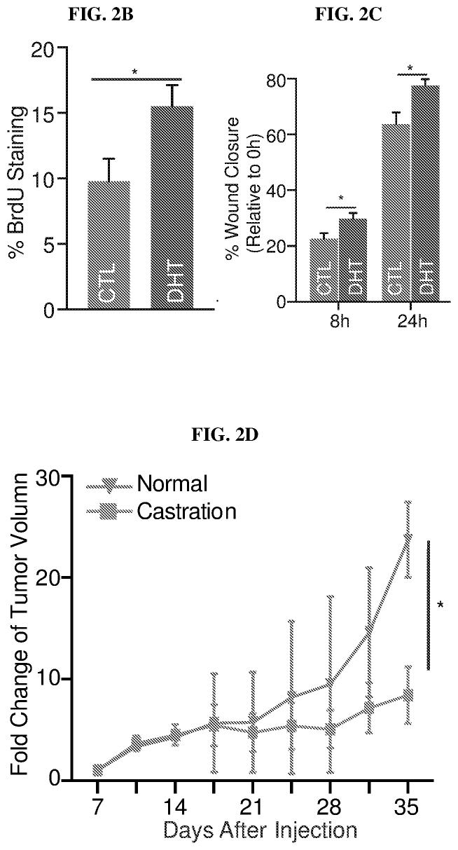

We show here that melanoma cells express androgen-inducible and transcriptionally active AR. AR expression is over 88% in both male and female melanoma patients (FIG. 1A), but is particularly increased in metastatic melanoma patients (FIG. 1B). As shown in FIG. 1D, Androgen stimulates the accumulation and nuclear translocation of androgen receptor in melanoma cells. Further, melanoma cells express variable levels of Androgen receptor (FIG. 1C). In these cells, androgen stimulates androgen receptor transcriptional activity (FIG. 1E). and this signaling drives melanoma proliferation, motility, and tumorigenesis (FIG. 2A-C). In fact, Androgen signaling is required for melanoma proliferation and motility. Through chemical castration, such as anti-androgen therapy, tumor volume is decreased (FIG. 2D).

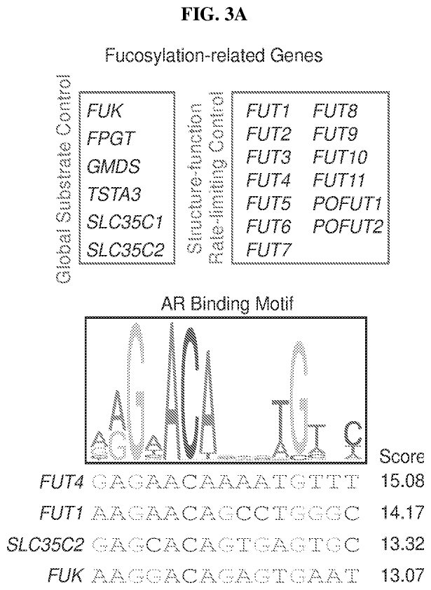

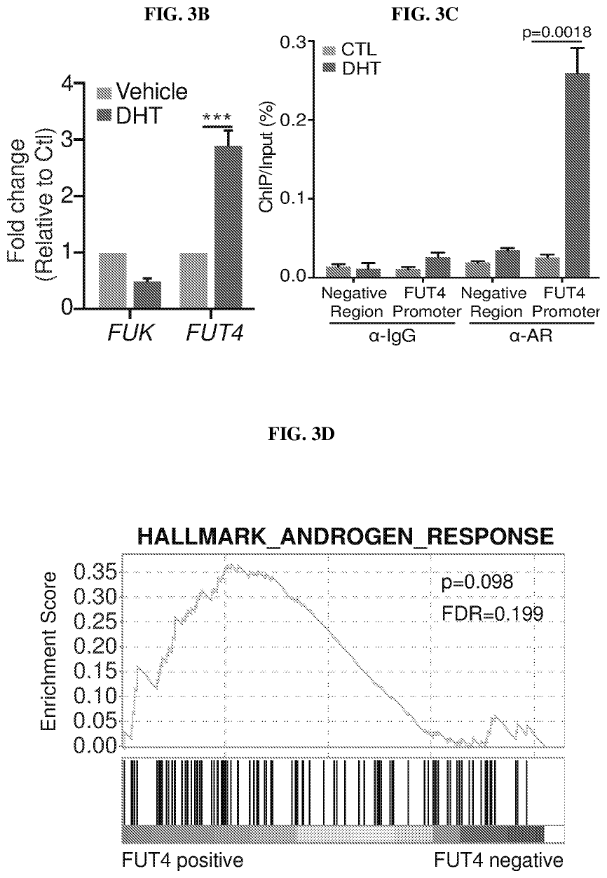

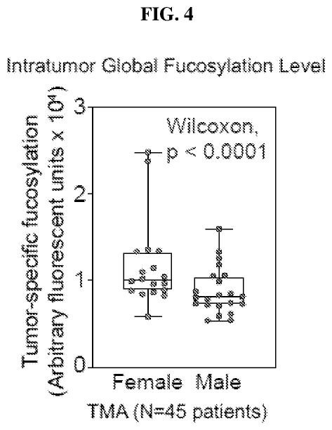

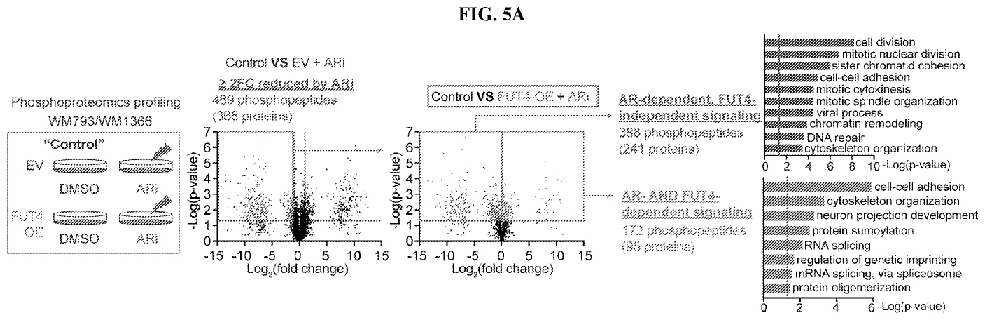

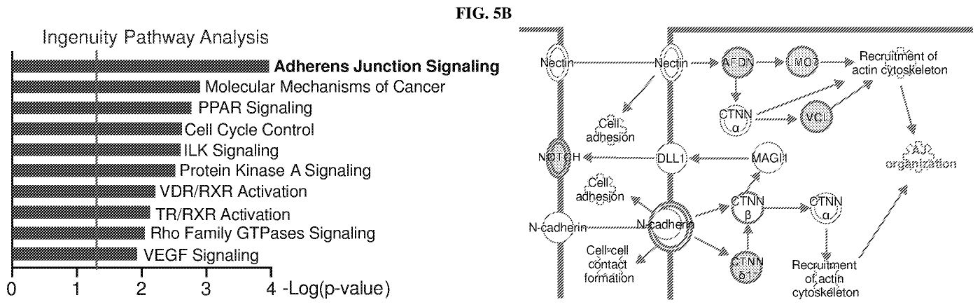

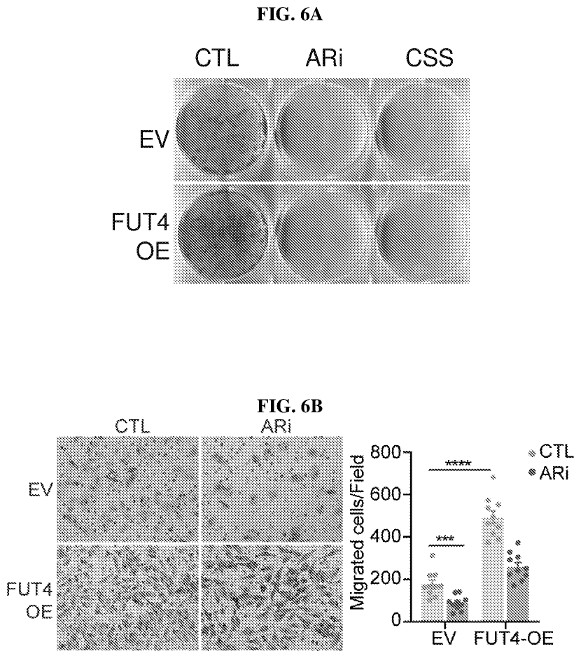

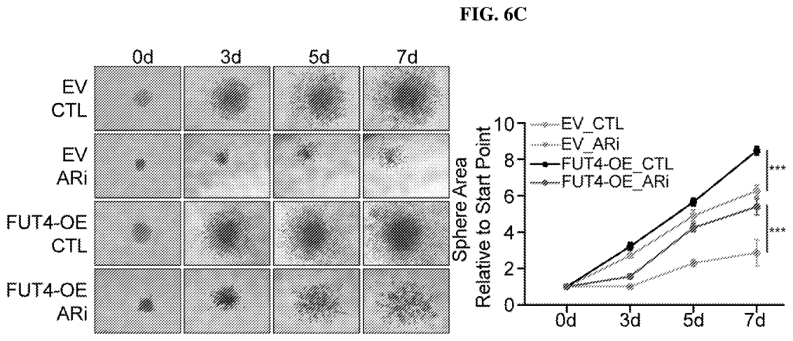

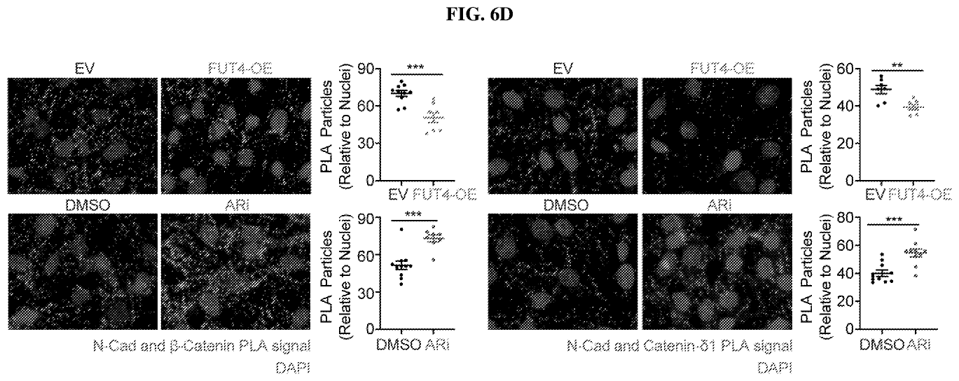

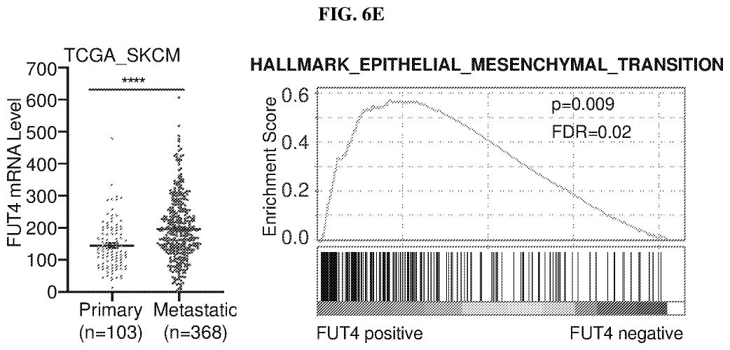

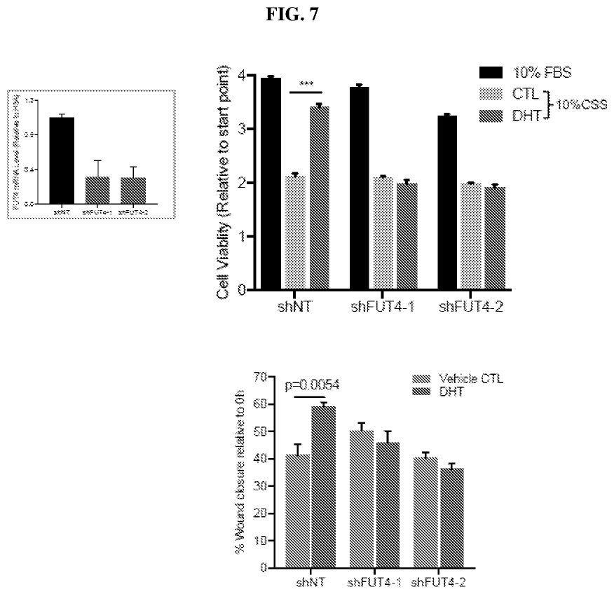

Global fucosylation is reduced during progression in human melanomas (UEA1 fucose-binding lectin staining analysis of tumor microarray (TMA; n=˜300 patients)) via an ATF2-mediated transcriptional repression of fucokinase (FUK). However, AR transcriptionally upregulates Fucosyltransferase 4 (FUT4) expression via binding to the ARE motif in FUT4 promoter (FIG. 3). Additionally, the global tumor fucosylation is more pronounced in males relative to females (FIG. 4). As shown in FIG. 5, AR-FUT4-dependent signaling regulates cell adhesion/motility, whereas AR-dependent/FUT4-independent signaling regulates cell division. Furthermore, the AR-FUT4 axis facilitates melanoma invasion via disrupting N-Cadherin/catenin junction complexes (FIG. 6). Knockdown experiments show that knockdown of FUT4 abrogates androgen stimulated melanoma activity. Thus, FUT4 is required for androgen-stimulated melanoma cell viability and motility (FIG. 7). Importantly, increasing fucosylation by genetic manipulation of tumor cells or by dietary L-fucose supplementation significantly blocks tumor growth and metastasis by >50% in mouse models.

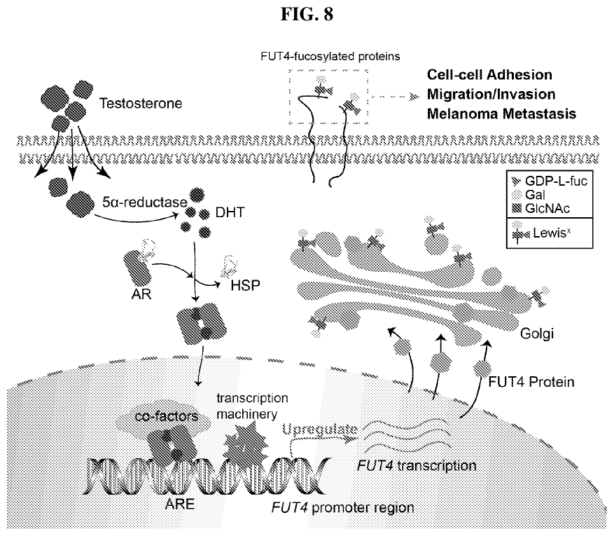

As depicted in FIG. 8, Melanoma cells express androgen-responsive AR, which is required for melanoma proliferation and migration. To accomplish this, the androgen receptor binds to DNA and regulates the FUT4 promoter. Androgen/AR promotes a tumorigenic subtype of fucosylation by transcriptionally upregulates FUT4 level via interacting with ARE motif in FUT4 promoter region. We shows that AR-FUT4 axis is responsible for facilitating melanoma invasiveness and metastatic spread. We further have demonstrated that Androgen/AR signaling regulates melanoma cell proliferation/division in a FUT4-independent manner. Also we show that the Androgen/AR/FUT4 axis triggers melanoma metastasis by disrupting N-cadherin-mediated cell-cell adhesion. The studies herein demonstrate that i) tumor fucosylation levels can be used to identify different stages of cancer, and ii), the manipulation of fucosylation represents a feasible anti-cancer approach.

It is understood and herein contemplated that, as evidenced by the work herein, the expression level of androgen receptor (AR), Fucosyltransferase 4 (FUT4), fucosylated L1CAM, FUT4 fucosylated/-regulated glycans and/or proteoglycans directly correlates with the motility and invasiveness of a cancer (such as, for example, melanoma, prostate cancer, or breast cancer) and/or metastasis (including, but not limited to metastatic melanoma, prostate cancer, or breast cancer). Thus, the levels further correlate with the severity of the cancer (such as, for example, melanoma, prostate cancer, or breast cancer). An increase in the expression level of androgen receptor (AR), Fucosyltransferase 4 (FUT4), fucosylated L1CAM, FUT4 fucosylated/-regulated glycans and/or proteoglycans relative to a control indicates that the cancer is metastatic. Similarly, a decrease or no change in the expression level of androgen receptor (AR), Fucosyltransferase 4 (FUT4), fucosylated L1CAM, FUT4 fucosylated/-regulated glycans and/or proteoglycans shows the cancer is not metastatic. Accordingly, in one aspect, disclosed herein are methods of detecting the presence or measuring the severity of a cancer and/or metastasis (such as, for example, melanoma, prostate cancer, or breast cancer) in a subject comprising obtaining a cancerous tissue sample from the subject and assaying the expression level of androgen receptor (AR), Fucosyltransferase 4 (FUT4), fucosylated L1CAM, FUT4 fucosylated/-regulated glycans and/or proteoglycans in the sample, wherein the presence of or an increase in the level of androgen receptor (AR), Fucosyltransferase 4 (FUT4), fucosylated L1CAM, FUT4 fucosylated/-regulated glycans and/or proteoglycans relative to a control indicates the presence of a cancer. Also disclosed herein are methods of measuring the severity of a cancer and/or metastasis (such as, for example, melanoma, prostate cancer, or breast cancer) in a subject comprising obtaining a cancerous tissue sample from the subject and assaying the level of androgen receptor (AR), Fucosyltransferase 4 (FUT4), fucosylated L1CAM, FUT4 fucosylated/-regulated glycans and/or proteoglycans in the sample, wherein the level of androgen receptor (AR), Fucosyltransferase 4 (FUT4), fucosylated L1CAM, FUT4 fucosylated/-regulated glycans and/or proteoglycans in the sample relative to a control directly correlates with the severity of the cancer. When an invasive or motile cancer is detected (i.e., a metastatic cancer and therefore a severe cancer), the method can further comprise the administration of an anti-androgen therapy in combination with an agent that increases fucosylation (such as, for example, L-fucose, D-fucose, fucose-1-phosphate, or GDP-L-fucose) to treat a cancer.

As the expression level of androgen receptor (AR), Fucosyltransferase 4 (FUT4), fucosylated L1CAM, FUT4 fucosylated/-regulated glycans and/or proteoglycans in cancerous tissue directly correlates with invasiveness and motility, it is understood and herein contemplated that the expression level of androgen receptor (AR), Fucosyltransferase 4 (FUT4), fucosylated L1CAM, FUT4 fucosylated/-regulated glycans and/or proteoglycans can then be used to assist in assigning treatment regimens to patients. In AR^{HI} cancers, Androgen/AR promotes a tumorigenic subtype of fucosylation by transcriptionally upregulates FUT4 level via interacting with ARE motif in FUT4 promoter region. This is particularly relevant as FUT4 is the primary driver of motility and invasiveness, but the action of FUT4 is dependent on the availability of fucose. Thus, while the administration of an agent that increases fucosylation (such as, for example, L-fucose, D-fucose, fucose-1-phosphate, or GDP-L-fucose) can be used to treat cancer, such agents will promote metastasis in AR+ cancers. Thus, an anti-androgen therapy is also needed in combination with the agent that increases fucosylation.

Accordingly, the expression level of androgen receptor (AR), Fucosyltransferase 4 (FUT4), fucosylated L1CAM, FUT4 fucosylated/-regulated glycans and/or proteoglycans can then be used as an indicator of which patients should receive anti-androgen therapy in combination with an agent that increases fucosylation (such as, for example, L-fucose, D-fucose, fucose-1-phosphate, or GDP-L-fucose) to treat a cancer. Accordingly, disclosed herein are methods of selecting cancer patients for anti-androgen therapy (including anti-androgen therapy in combination with an agent that increases fucosylation (such as, for example, L-fucose, D-fucose, fucose-1-phosphate, or GDP-L-fucose)) comprising obtaining a cancerous tissue sample from the patient and measuring the level of androgen receptor (AR), Fucosyltransferase 4 (FUT4), fucosylated L1CAM, FUT4 fucosylated/-regulated glycans and/or proteoglycans in the tissue sample, wherein an increase in FUT4 and/or FUT4 fucosylated/-regulated glycans and/or proteoglycans relative to noncancerous tissue indicates that the patient should receive anti-androgen therapy (including anti-androgen therapy in combination with an agent that increases fucosylation (such as, for example, L-fucose, D-fucose, fucose-1-phosphate, or GDP-L-fucose).

In one aspect, disclosed herein are methods of treating a treating, inhibiting, decreasing, reducing, ameliorating, and/or preventing a cancer (such as, for example, melanoma, prostate cancer, or breast cancer) and/or metastasis (such as, for example, metastatic melanoma, prostate cancer, or breast cancer) in a subject comprising administering to the subject an agent (such as, for example, L-fucose, D-fucose, fucose-1-phosphate, or GDP-L-fucose) that increases the amount of fucosylation and an anti-androgen therapy. In some aspect, the method can further comprise detecting the level of androgen receptor (AR), Fucosyltransferase 4 (FUT4), fucosylated L1CAM, FUT4 fucosylated/-regulated glycans and/or proteoglycans in a cancerous tissue sample from the subject prior to administration of the agent (such as, for example, L-fucose, D-fucose, fucose-1-phosphate, or GDP-L-fucose) that increases the amount of fucosylation and an anti-androgen therapy.

Anti-androgen therapies are well known in the art including but not limited to luteinizing hormone-releasing hormone (LHRH) agonists (such as, for example, leuprolide, goserelin, triptorelin, and/or histrelin), LHRH antagonist (such as, for example degarelix and/or relugolix), and/or anti-androgen therapy (such as, for example, bicalutamide, nilutamide, flutamide, abiraterone, corticosteroids, ketoconazole, and/or apalutamide). Thus, in one aspect, disclosed herein are methods of treating a treating, inhibiting, decreasing, reducing, ameliorating, and/or preventing a cancer and/or metastasis (such as, for example, melanoma, prostate cancer, or breast cancer) in a subject comprising administering to the subject an agent (such as, for example, L-fucose, D-fucose, fucose-1-phosphate, or GDP-L-fucose) that increases the amount of fucosylation and an anti-androgen therapy (anti-androgen therapies including but not limited to luteinizing hormone-releasing hormone (LHRH) agonists (such as, for example, leuprolide, goserelin, triptorelin, and/or histrelin), LHRH antagonist (such as, for example degarelix and/or relugolix), and/or anti-androgen therapy (such as, for example, bicalutamide, nilutamide, flutamide, abiraterone, corticosteroids, ketoconazole, and/or apalutamide).

As shown herein, administration of an agent (such as, for example, L-fucose, D-fucose, fucose-1-phosphate, or GDP-L-fucose) makes myeloid derived suppressor cells immunostimulatory and results in increased immune cells in the tumor microenvironment. The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the fucose comprising compositions into the nose and nasal passages through one or both of the nares and can comprise delivery by a spraying mechanism or droplet mechanism, or through aerosolization of the nucleic acid or vector. Administration of the fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) by inhalant can be through the nose or mouth via delivery by a spraying or droplet mechanism. Delivery can also be directly to any area of the respiratory system (e.g., lungs) via intubation. The exact amount of the fucose comprising compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions), if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions) can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The fucose comprising compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Fucose modulating compositions (including, but not limited to fucose (such as, for example L-fucose, D-fucose, fucoidan, fucose-1-phosphate, GDP-L-fucose, or L-fucose/GDP-L-fucose analogues) and fucose comprising compositions for oral administration include powders or granules, suspensions or solutions in water or non-aqueous media, capsules, sachets, or tablets. Thickeners, flavorings, diluents, emulsifiers, dispersing aids or binders may be desirable.

Some of the fucose modulating compositions (including, but not limited to fucose (such as, for example L-fucose, D-fucose, fucose-1-phosphate, or GDP-L-fucose) and fucose comprising compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the fucose comprising compositions may be determined empirically, and making such determinations is within the skill in the art. The dosage ranges for the administration of the fucose comprising compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

As disclosed herein, administration of the fucose or fucose comprising composition can occur at any time before, during, or after administration of anti-androgens including at least 96, 84, 72, 60, 48, 36, 24, 18, 12, 8, 6, 5, 4, 3, 2, 1 hrs, 45, 30, 15, 10, or 5 minutes before or at least 1, 2, 3, 4, 5, 10, 15, 30, 45 minutes, 1, 2, 3, 4, 5, 6, 7, 8, 9, 10, 11, 12, 18, 24, 36, 48, 60, 72, 84, or 96 hours after anti-androgen administration or concurrent with the commencement of anti-androgens.

Administration of anti-androgens and the agent that modulates fucosylation can also occur in conjunction with adaptive therapy. Specifically, administration of the fucose or fucose comprising composition and anti-androgen therapy can occur at any time before, during, or after production of TILs, MILs, and/or CAR T cells including, but not limited to before, during, or after pre-REP or before, during, or after REP. In other words, administration of fucose and anti-androgen therapy can occur before pre-REP can occur at least 96, 84, 72, 60, 48, 36, 24, 18, 12, 8, 6, 5, 4, 3, 2, 1 hrs, 45, 30, 15, 10, or 5 minutes before the pre-REP expansion, concurrent with the commencement of pre-REP expansion, or at least 1, 2, 3, 4, 5, 10, 15, 30, 45 minutes, 1, 2, 3, 4, 5, 6, 7, 8, 9, 10, 11, 12, 18, 24, 36, 48, 60, 72, 84, or 96 hours after the commencement of the pre-REP expansion. Similarly, administration of fucose can occur before REP expansion can occur at least 96, 84, 72, 60, 48, 36, 24, 18, 12, 8, 6, 5, 4, 3, 2, 1 hrs, 45, 30, 15, 10, or 5 minutes before the REP expansion, concurrent with the commencement of pre-REP expansion, or at least 1, 2, 3, 4, 5, 10, 15, 30, 45 minutes, 1, 2, 3, 4, 5, 6, 7, 8, 9, 10, 11, 12, 18, 24, 36, 48, 60, 72, 84, or 96 hours after the commencement of the REP expansion. In one aspect, fucose and anti-androgen therapy can be administered to the subject in vivo following REP expansion of TILS and before, concurrently with, or after administration of TILs grown ex vivo are transferred to a subject in need thereof.

In one aspect, it is understood and herein contemplated that administration of an agent that increases fucosylation (such as, for example, L-fucose, D-fucose, fucose-1-phosphate, or GDP-L-fucose) and an anti-androgen therapy (anti-androgen therapies including but not limited to luteinizing hormone-releasing hormone (LHRH) agonists (such as, for example, leuprolide, goserelin, triptorelin, and/or histrelin), LHRH antagonist (such as, for example degarelix and/or relugolix), and/or anti-androgen therapy (such as, for example, bicalutamide, nilutamide, flutamide, abiraterone, corticosteroids, ketoconazole, and/or apalutamide) alone may not be sufficient to control a cancer. Thus, disclosed herein are methods of treating a treating, inhibiting, decreasing, reducing, ameliorating, and/or preventing a cancer and/or metastasis (such as, for example, melanoma) in a subject comprising administering to the subject fucose (such as for example, L-fucose, D-fucose, fucoidan, fucose-1-phosphate, GDP-L-fucose, or L-fucose/GDP-L-fucose analogues) and an anti-androgen therapy, further comprising the administration of an anti-cancer agent or immune checkpoint inhibitor (such as, for example, PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab) or any other anti-cancer agent disclosed herein), adoptive cell therapies, and/or CAR T therapies.

The disclosed methods of treating, inhibiting, reducing, decreasing, ameliorating, and/or preventing cancer and/or metastasis can be used to treat any disease where uncontrolled cellular proliferation occurs such as cancers. Thus, in one aspect disclosed herein are methods of treating, inhibiting, decreasing, reducing, ameliorating, and/or preventing a cancer and/or metastasis (such as, for example, a melanoma) in a subject comprising administering to the subject an agent that an agent that modulates (including increases) the amount of fucosylation on the cell (such as a fucose including, but not limited to L-fucose, D-fucose, fucoidan, fucose-1-phosphate, GDP-L-fucose, or L-fucose/GDP-L-fucose analogues) and an anti-androgen therapy (anti-androgen therapies including but not limited to luteinizing hormone-releasing hormone (LHRH) agonists (such as, for example, leuprolide, goserelin, triptorelin, and/or histrelin), LHRH antagonist (such as, for example degarelix and/or relugolix), and/or anti-androgen therapy (such as, for example, bicalutamide, nilutamide, flutamide, abiraterone, corticosteroids, ketoconazole, and/or apalutamide). A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer. The methods disclosed herein may also be used for the treatment of precancer conditions such as cervical and anal dysplasias, other dysplasias, severe dysplasias, hyperplasias, atypical hyperplasias, and neoplasias. As noted herein, the disclosed methods are particularly useful in cancers that are highly immunosuppressive. Accordingly, it is understood and herein contemplated that the disclosed methods of treatment can further comprise first determining if the cancer being treated in highly immunosuppressive. Thus, in one aspect, disclosed herein are methods of treating a treating, inhibiting, decreasing, reducing, ameliorating, and/or preventing a cancer and/or metastasis in a subject, further comprising detecting whether the cancer has high levels of FUT4 and/or FUT4 fucosylated/-regulated glycans and/or proteoglycans prior to administration of L-fucose and/or anti-androgen therapy.

As noted above, the disclosed methods of treating a cancer with an agent that an agent that modulates (including increases) the amount of fucosylation on the cell (such as a fucose including, but not limited to L-fucose, D-fucose, fucoidan, fucose-1-phosphate, GDP-L-fucose, or L-fucose/GDP-L-fucose analogues) and an anti-androgen therapy (anti-androgen therapies including but not limited to luteinizing hormone-releasing hormone (LHRH) agonists (such as, for example, leuprolide, goserelin, triptorelin, and/or histrelin), LHRH antagonist (such as, for example degarelix and/or relugolix), and/or anti-androgen therapy (such as, for example, bicalutamide, nilutamide, flutamide, abiraterone, corticosteroids, ketoconazole, and/or apalutamide) contemplate the co-administration of a CD4+ T cell mediated therapy such as an anti-cancer agent. The anti-cancer agent can comprise any anti-cancer agent known in the art including, but not limited to antibodies, tumor infiltrating lymphocytes, checkpoint inhibitors, dendritic cell vaccines, anti-cancer vaccines, immunotherapy, and chemotherapeutic agents. In one aspect, the anti-cancer agent can include, but is not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), OPDIVO (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Also contemplated herein are chemotherapeutics that are checkpoint inhibitors, such as, for example, PD1/PDL1 blockade inhibitors and/or CTLA4/B7-1 or 2 inhibitors (such as, for example, PD-1 inhibitors lambrolizumab, OPDIVO® (Nivolumab), KEYTRUDA® (pembrolizumab), and pidilizumab; PD-L1 inhibitors BMS-936559, TECENTRIQ® (Atezolizumab), IMFINZI® (Durvalumab), and BAVENCIO® (Avelumab); and CTLA-4 inhibitors YERVOY (ipilimumab). In one aspect, the CD4+ T cell mediated therapy can comprise adoptive cell therapies, and CAR T therapies. Accordingly, disclosed herein are methods of treating, inhibiting, decreasing, reducing, ameliorating, and/or preventing a cancer or metastasis (such as, for example, a melanoma) in a subject comprising administering to the subject i) an immune checkpoint blockade inhibitor (such as, for example, the PD-1 inhibitors lambrolizumab, OPDIVO® (Nivolumab), KEYTRUDA® (pembrolizumab), and/or pidilizumab; the PD-L1 inhibitors BMS-936559, TECENTRIQ® (Atezolizumab), IMFINZI® (Durvalumab), and/or BAVENCIO® (Avelumab); and/or the CTLA-4 inhibitor YERVOY (ipilimumab)) and ii) an agent that an agent that modulates (including increases) the amount of fucosylation on the cell (such as a fucose including, but not limited to L-fucose, D-fucose, fucoidan, fucose-1-phosphate, GDP-L-fucose, or L-fucose/GDP-L-fucose analogues). In one aspect, the fucose can be administered before and/or during administration of the anti-cancer agent.

The combination of fucose and an anti-cancer agent or immune checkpoint inhibitor can be formulated in the same composition of separately. Where separate, the fucose can be administered before, after, or concurrently with the anti-cancer agent. Administration of fucose can be accomplished prophylactically or therapeutically.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

1. A method of treating a treating, inhibiting, decreasing, reducing, ameliorating, and/or preventing a cancer and/or metastasis in a subject comprising administering to the subject an agent that increases the amount of fucosylation and an anti-androgen therapy.

2. The method of treating a treating, inhibiting, decreasing, reducing, ameliorating, and/or preventing a cancer and/or metastasis in a subject of claim 1, wherein the agent that increases fucosylation comprises L-fucose, D-fucose, fucose-1-phosphate, or GDP-L-fucose.

3. The method of treating a treating, inhibiting, decreasing, reducing, ameliorating, and/or preventing a cancer and/or metastasis in a subject of claim 1, wherein the agent that modulates fucosylation is administered orally.

4. The method of treating a treating, inhibiting, decreasing, reducing, ameliorating, and/or preventing a cancer and/or metastasis in a subject of claim 1, wherein the anti-androgen therapy comprises a Luteinizing hormone-releasing hormone (LHRH) agonists, LHRH antagonist, anti-androgen therapy.

5. The method of treating a treating, inhibiting, decreasing, reducing, ameliorating, and/or preventing a cancer and/or metastasis in a subject of claim 4, wherein the anti-androgen therapy comprises administration of leuprolide, goserelin, triptorelin, histrelin, degarelix, relugolix, bicalutamide, nilutamide, flutamide, abiraterone, corticosteroids, ketoconazole, and/or apalutamide.

6. The method of treating a treating, inhibiting, decreasing, reducing, ameliorating, and/or preventing a cancer and/or metastasis in a subject of claim 1, wherein the fucose is administered before, after, concurrent to and/or simultaneously with the administration of the anti-androgen therapy.

7. The method of treating a treating, inhibiting, decreasing, reducing, ameliorating, and/or preventing a cancer and/or metastasis in a subject of claim 1 further comprising administering to the subject an immune checkpoint blockade inhibitor.

8. The method of treating a treating, inhibiting, decreasing, reducing, ameliorating, and/or preventing a cancer and/or metastasis in a subject of claim 7, wherein the immune checkpoint blockade inhibitor is selected from the group consisting of the PD-1 inhibitors lambrolizumab, OPDIVO® (Nivolumab), KEYTRUDA® (pembrolizumab), and/or pidilizumab; the PD-L1 inhibitors BMS-936559, TECENTRIQ® (Atezolizumab), IMFINZI® (Durvalumab), and/or BAVENCIO® (Avelumab); and/or the CTLA-4 inhibitor YERVOY (ipilimumab).

9. The method of treating a treating, inhibiting, decreasing, reducing, ameliorating, and/or preventing a cancer and/or metastasis in a subject of claim 1 further comprising administering to the subject an adoptive cell therapy.

10. The method of treating a treating, inhibiting, decreasing, reducing, ameliorating, and/or preventing a cancer and/or metastasis in a subject of claim 9, wherein the adoptive cell therapy comprises the transfer of tumor infiltrating lymphocytes (TILs), tumor infiltrating NK cells (TINKs), marrow infiltrating lymphocytes (MILs), chimeric antigen receptor (CAR) T cells, and/or CAR NK cells.

11. The method of treating a treating, inhibiting, decreasing, reducing, ameliorating, and/or preventing a cancer and/or metastasis in a subject of claim 1, wherein cancer is a melanoma.

12. The method of treating a treating, inhibiting, decreasing, reducing, ameliorating, and/or preventing a cancer and/or metastasis in a subject of claim 1, further comprising detecting the level of androgen receptor (AR), Fucosyltransferase 4 (FUT4), fucosylated L1CAM, FUT4 fucosylated/-regulated glycans and/or proteoglycans prior to administration of an anti-androgen or an agent that modulates fucosylation; wherein an increase in androgen receptor (AR), Fucosyltransferase 4 (FUT4), fucosylated L1CAM, FUT4 fucosylated/-regulated glycans and/or proteoglycans indicates that the anti-androgen and the agent that modulates fucosylation should be administered.

13. A method of detecting the presence of a cancer and/or metastasis in a subject comprising obtaining a tissue sample from the subject and assaying the level of androgen receptor (AR), Fucosyltransferase 4 (FUT4), fucosylated L1CAM, FUT4 fucosylated/-regulated glycans and/or proteoglycans in the sample, wherein the presence of or an increase in the level of androgen receptor (AR), Fucosyltransferase 4 (FUT4), fucosylated L1CAM, FUT4 fucosylated/-regulated glycans and/or proteoglycans relative to a control indicates the presence of a cancer and/or metastasis or a severe cancer and/or metastasis.

14. A method of measuring the severity of a cancer and/or metastasis in a subject comprising obtaining a cancerous tissue sample from the subject and assaying the level of androgen receptor (AR), Fucosyltransferase 4 (FUT4), fucosylated L1CAM, FUT4 fucosylated/-regulated glycans and/or proteoglycans in the sample, wherein the level of androgen receptor (AR), Fucosyltransferase 4 (FUT4), fucosylated L1CAM, FUT4 fucosylated/-regulated glycans and/or proteoglycans in the sample relative to a control directly correlates with the severity of the cancer.

15. A method of selecting cancer patient for anti-androgen therapy comprising obtaining a cancerous tissue sample from the patient and measuring the level of androgen receptor (AR), Fucosyltransferase 4 (FUT4), fucosylated L1CAM, FUT4 fucosylated/-regulated glycans and/or proteoglycans in the tissue sample, wherein an increase in androgen receptor (AR), Fucosyltransferase 4 (FUT4), fucosylated L1CAM, FUT4 fucosylated/-regulated glycans and/or proteoglycans relative to noncancerous tissue indicates that the subject should receive anti-androgen therapy.

16. The method of selecting cancer patient for anti-androgen therapy of claim 15, wherein the method further comprises administering to the patient an agent that increases fucosylation.


SEQUENCE LISTING (4 sequences)

SEQ ID NO: 1 - DNA, 15 bp, Artificial Sequence
  Synthetic construct
gagaacaaaatgttt

SEQ ID NO: 2 - DNA, 15 bp, Artificial Sequence
  Synthetic construct
aagaacagcctgggc

SEQ ID NO: 3 - DNA, 15 bp, Artificial Sequence
  Synthetic construct
gagcacagtgagtgc

SEQ ID NO: 4 - DNA, 15 bp, Artificial Sequence
  Synthetic construct
aaggacagagtgaat